(19) United States
(12) Patent Application Publication
(10) Pub. No.: US 2026/0306377 A1
(43) Pub. Date: Oct. 8, 2026

(54) ANTIMICROBIAL COMPOSITIONS CONTAINING A CATIONIC ANTIMICROBIAL AND AN ACIDIC GLYCOLOPID

(71) Applicant: LANXESS CORPORATION, Pittsburgh, PA (US)

(72) Inventors: Siri Tan, Gloucester City, NJ (US); Marcelo Filgueira, Kennett Square, PA (US); David Laganella, Blackwood, NJ (US)

(21) Appl. No.: 19/476,405

(22) PCT Filed: Apr. 22, 2024
(86) PCT No.: PCT/US2024/025662

§ 371 Date: Oct. 17, 2025

Related U.S. Application Data
(60) Provisional application No. 63/498,515, filed on Apr. 26, 2023.

(51) Int. Cl. A61K 8/44; A61K 8/43

(52) U.S. Cl. CPC A61K 8/442; A61K 8/43; A61K 2800/524

(57) ABSTRACT

The present disclosure relates to antimicrobial compositions containing at least one cationic antimicrobial and at least one acidic glycolipid and the use of such compositions in product formulations, particularly anionic-type formulations, such as in personal care, home care and other product formulations.

### FIELD OF INVENTION

The present invention relates to antimicrobial compositions containing a cationic antimicrobial and an acidic glycolipid and the use of such compositions in product formulations, particularly anionic formulations, such as in personal care, home care and other product formulations.

### BACKGROUND OF THE INVENTION

Many cationic substances, such as ethyl lauroyl arginate hydrochloride (LAE), chlorhexidine digluconate, and others, are known to be effective as antimicrobial agents and preservatives. However, many product formulations are based on anionic formulations, such as formulations employing anionic rheology modifiers, anionic surfactants and/or other anionic substances. Cationic antimicrobial agents, such as LAE, are generally not compatible with such anionic formulation components, the combination of which can often lead to aggregation and/or turbidity and diminished antimicrobial activity. Cationic antimicrobials are therefore generally unsuitable for use in anionic formulations. For example, while LAE is promoted as a broad spectrum, food safe preservative and is used in many personal care, food, and other product formulations, it is known to exhibit incompatibilities with anionic thickeners, such as carbomer, carboxymethyl cellulose, carrageenan and xanthan gum, and anionic surfactants, such as sodium laureth or lauryl sulfate (SLES/SLS) and alkyl phosphates and carboxylates. Use in anionic formulations is typically avoided, particularly as the efficacy of cationic antimicrobials, such as LAE, is generally lost.

Consequently, there is a need, such as in the personal care, home care and other industries, to compatibilize cationic antimicrobials for use in anionic product formulations and to achieve efficacious antimicrobial activity therein.

This need is met by the present disclosure. It was discovered that using cationic antimicrobials in combination with acidic glycolipids as described herein enables the cationic antimicrobials to be used in anionic formulations as effective antimicrobial agents and preservatives. Additionally, combining the cationic antimicrobials with such glycolipids allows for efficient antimicrobial replacement solutions in anionic product formulations, such as in personal care (e.g., cosmetics), home care and other industries, and can further provide efficacious antimicrobial activity in product formulations, whether anionic, nonionic or cationic, at lower use levels of the cationic antimicrobials than previously reported for such substances.

### SUMMARY OF INVENTION

Provided is an antimicrobial composition comprising a mixture of (a) at least one cationic antimicrobial and (b) at least one acidic glycolipid, wherein the amount by weight of the at least one acidic glycolipid (b) is greater than or about equal to the amount by weight of the at least one cationic antimicrobial (a).

Also provided is a method of preparing an antimicrobial composition comprising combining (a) at least one cationic antimicrobial and (b) at least one acidic glycolipid, wherein the amount by weight of the at least one acidic glycolipid (b) is greater than or about equal to the amount by weight of the at least one cationic antimicrobial (a). Preferably, the components (a) and (b) are combined in water and/or a water-miscible solvent. Preferably, the components (a) and (b) are combined prior to use in or addition to a product formulation, especially an anionic product formulation.

In another aspect, provided is a product formulation, such as for use as a personal care, home care, health care, food, or industrial product, comprising an effective amount of the antimicrobial composition of the present disclosure.

The product formulation of the present disclosure is preferably an anionic formulation, e.g., an anionic product formulation comprising at least one anionic rheology modifier, at least one anionic surfactant, at least one anionic emulsifier or any combination thereof, and an antimicrobial effective amount of a combination of at least one cationic antimicrobial or preservative and at least one acidic glycolipid, wherein the amount by weight of the at least one acidic glycolipid is greater than or about equal to the amount by weight of the at least one cationic antimicrobial or preservative.

Also provided is a method of preparing or formulating a product formulation, such as for use as a personal care, home care, health care, food, or industrial product, comprising adding an effective amount of (a) at least one cationic antimicrobial and (b) at least one acidic glycolipid to the product formulation, wherein the amount by weight of the at least one acidic glycolipid (b) is greater than or about equal to the amount by weight of the at least one cationic antimicrobial (a). The components (a) and (b) may be added to the product formulation individually or in the form of a mixture, preferably in the form of a mixture, such as described herein for the antimicrobial composition, especially for addition to anionic product formulations. Preferably, the product formulation is an anionic formulation.

In a further aspect, provided is a method of controlling or inhibiting microbial growth in or preserving a product formulation, such as a formulation for use as a personal care, home care, health care, food or industrial product, comprising adding an effective amount of (a) at least one cationic antimicrobial and (b) at least one acidic glycolipid to the product formulation, wherein the amount by weight of the at least one acidic glycolipid (b) is greater than or about equal to the amount by weight of the at least one cationic antimicrobial (a). The components (a) and (b) may be added to the product formulation individually or in the form of a mixture, preferably in the form of a mixture, such as described herein for the antimicrobial composition, especially for addition to anionic product formulations. Preferably, the product formulation is an anionic formulation. The method may further include preparing the antimicrobial composition by combining the at least one cationic antimicrobial (a) and the at least one acidic glycolipid (b), typically in water and/or a water-miscible solvent, prior to adding the antimicrobial composition to a product formulation, especially an anionic formulation.

Further provided is a method of preparing or formulating at least one cationic antimicrobial for use in an anionic product formulation, the method comprising combining the at least one cationic antimicrobial with an acidic glycolipid, typically in water and/or a water-miscible solvent, prior to use or addition of the cationic antimicrobial in an anionic product formulation, wherein the amount by weight of the at least one acidic glycolipid is greater than or about equal to the amount by weight of the at least one cationic antimicrobial.

### DETAILED DESCRIPTION

Unless otherwise specified, the following terms are defined as follows:

As used herein, “formulation” refers to a preparation that is to be preserved or provided with antimicrobial activity using the antimicrobial composition of the present disclosure.

As used herein, a “cationic antimicrobial” is an antimicrobial compound having one or more cationic groups. Common examples in accordance with the present disclosure are antimicrobial compounds having one or more cationic nitrogen-based groups.

As used herein, “anionic formulation” means a formulation employing one or more anionic compounds. Examples of anionic compounds include, but are not limited to, anionic rheology modifiers and anionic surfactants, such as anionic thickeners and anionic emulsifiers.

The term “glycolipid” as used herein means a compound comprising a carbohydrate group having one or more monosaccharide residues (e.g., monosaccharide, disaccharide, oligosaccharide or polysaccharide residues) bound by a glycosidic linkage to a lipid group.

The term “glycosidic bond” or “glycosidic linkage” as used herein refers to a covalent bond linking a monosaccharide or monosaccharide residue of the carbohydrate component to the lipid component.

In the context of the presently disclosed antimicrobial composition, “an acidic glycolipid” means at least one glycolipid in an acidic form or salt thereof (with respect to the salt, e.g., a physiologically, cosmetically, pharmaceutically or nutraceutically acceptable salt), that is, the lipid component of the glycolipid contains at least one carboxylic acid group in the form of a fatty acid moiety or salt thereof, and the carbohydrate component is bound by a glycosidic linkage to the lipid component but not via the carboxylic acid group. In general, the acidic glycolipid is typically water miscible and/or water soluble.

As used herein, “effective amount” refers to an amount to provide a desired antimicrobial effect or activity.

In accordance with the present disclosure, it was surprisingly discovered that combining acidic glycolipids with cationic antimicrobials enables the cationic antimicrobials to be used in anionic formulations as effective antimicrobial agents and preservatives. It was additionally unexpectedly found that the combination with an acidic glycolipid can allow for the use of reduced levels of the cationic antimicrobial in product formulations, whether anionic, nonionic or cationic, compared to previously reported levels to achieve efficacious antimicrobial activity.

The cationic antimicrobial of the present disclosure may be, for example, an antimicrobial compound having one or more cationic nitrogen-based groups. Examples of such compounds are known in the art. For example, the cationic antimicrobial is preferably chosen from N^{α}-alkanoyl dibasic amino acid ester salts, such as N^{α}-alkanoyl dibasic amino acid alkyl ester salts. In many such embodiments, the cationic antimicrobial is a N^{α}—(C₆-C₂₀)alkanoyl dibasic amino acid (C₁-C₆)alkyl ester salt. The N^{α}-alkanoyl dibasic amino acid ester salt is preferably derived from a dibasic amino acid ester, such as an alkyl ester, of L-arginine, L-histidine, L-tryptophan, or L-lysine, more preferably L-arginine. The cationic antimicrobial may be derived from the condensation of fatty acids, such as C₆-C₂₀ fatty acids, and esterified dibasic amino acids, such as the amino acids described above. Preferably, the N^{α}-alkanoyl dibasic amino acid ester salt is derived from N^{α}—(C₆-C₂₀)alkanoyl-L-arginine (C₁-C₆)alkyl ester, such as N^{α}-lauroyl-L-arginine (C₁-C₆)alkyl ester, more particularly N^{α}-lauroyl-L-arginine ethyl ester.

The choice of anionic component of the N^{α}-alkanoyl dibasic amino acid ester salt is not limited to a particular anion and can be chosen such that, for example, the solubility of the salt is sufficient for the intended mixture with the acidic glycolipid and may also account for intended application of the antimicrobial composition. Examples of such anions include, but are not limited to, halide, linolenate, laurate, oleoate, nitrate, nitrite, gluconate, and pyroglutamate.

The N^{α}-alkanoyl dibasic amino acid ester salt is preferably of the formula (I)

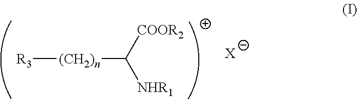

where R₁ is a straight alkyl chain from a saturated fatty acid or a hydroxy acid having from 6 to 20, preferably from 8 to 14, carbon atoms bonded to the α-amino group through an amidic bond,
R₂ is a straight or branched alkyl chain from 1 to 6 carbon atoms, such as from 1 to 4, or from 1 to 2, carbon atoms, or an aromatic group,
R₃ is selected from

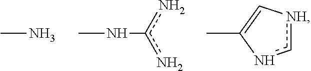

n is from 1 to 6, and
X⁻ is Cl⁻, Br⁻ or a counter ion derived from an organic or inorganic acid or a phenolic compound.

Examples of acids that may be the source of the counter ion X include acetic acid, citric acid, lactic acid, fumaric acid, maleic acid, gluconic acid, propionic acid, sorbic acid, benzoic acid, carbonic acid, glutamic acid, pyroglutamic acid, lauric acid, oleic acid, linoleic acid, phosphoric acid, nitric acid, sulfuric acid, and thiocyanic acid. Examples of phenolic compounds that may be the source of the counter ion X include butylated hydroxyanisole (BHA), butylated hydroxytoluene, tertiary butylhydroquinone, methylparaben, ethylparaben, propylparaben, and butylparaben.

The N^{α}-alkanoyl dibasic amino acid alkyl ester salt is more preferably of the formula (II)

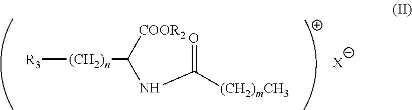

where R₂, R₃, n and X are as above in formula (I), and
m is from 6 to 18 preferably from 6 to 12.

A particular example of the N^{α}-alkanoyl dibasic amino acid ester salt is ethyl lauroyl arginate HCl (LAE), which may be prepared from L-arginine and lauric acid, in particular, the ethyl ester of the lauramide of arginine monohydrochloride. The chemical structure of LAE is shown in formula (III)

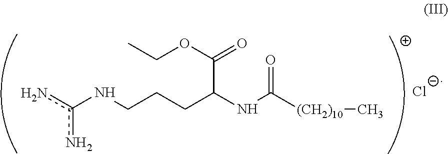

Another example of the N^{α}-alkanoyl dibasic amino acid ester salt contains the N^{α}-cocoyl-L-arginine ethyl ester cation, such as CAE (pyrrolidone carboxylic acid (PCA) ethyl cocoyl arginate).

In preferred embodiments, the cationic antimicrobial has one or more cationic guanidine or amidine functional groups. LAE and CAE described above are examples of cationic antimicrobials having a cationic guanidine group. Further examples include, but are not limited to, alkyl-guanidines and salts thereof, such as dodecylguanidine and salts thereof (e.g., hydrochloride salt), biguanides and salts thereof, such as chlorhexidine, alexidine, and salts thereof (e.g., digluconate, diacetate, dihydrochloride, cyclamate, etc.), and polybiguanides and salts thereof, such as polyhexamethylene biguanide (PHMB), polyaminopropyl biguanide and salts thereof (e.g., hydrochloride, acetate, gluconate, etc.). Hexamidine and salts thereof (e.g., diisethionate) are examples of cationic antimicrobials containing cationic amidine functional groups. Further examples of cationic antimicrobials having one or more guanidine or amidine functional groups, including further examples of the foregoing subgenera, are known in the art.

The cationic antimicrobial may be a quaternary ammonium compound, including salts thereof. Examples include, but are not limited to, benzalkonium, cetrimonium, cetylpyridinium, alkyltrimethyl ammonium, dialkyldimethyl ammonium, polyquaternium and salts thereof (e.g., chloride, bromide, etc.). Further examples of quaternary ammonium antimicrobials, as well as of the foregoing subgenera, are known in the art.

The cationic antimicrobial may further be chosen from peptides, such as polylysine (e.g., ε-poly-L-lysine), nisin, natamycin, etc.

More than one cationic antimicrobial may be used in the compositions, formulations and methods of the present disclosure.

As to the acidic glycolipid of the present disclosure, in many embodiments the acidic glycolipid includes a monosaccharide, disaccharide or oligosaccharide moiety. In many embodiments, the acidic glycolipid includes one, two or three monosaccharide residues. The one or more monosaccharide residues of the acidic glycolipid may be derived, for example, from glucose, sucrose, fructose, xylose, galactose, rhamnose, arabinose, mannose, cellobiose, lactose, galacturonate, sophorose or a mixture thereof. One or more of the hydroxyl groups of the monosaccharide residues may be acylated (e.g., acetylated) or etherified. Regarding the lipid component, the fatty acid moiety may be saturated or unsaturated and may contain, for example, from 6 to 30 carbon atoms (e.g., from 8 to 28 or from 10 to 26 carbon atoms), which may be linear or branched. The fatty acid chains may be substituted with functional groups, which are not particularly limited, provided that in general the acidic glycolipid is typically water-miscible and/or water soluble. Examples of substituents include, but are not limited to, halogen atoms, hydroxyl, alkyl groups (e.g., C₁₋₆ alkyl), halo alkyl groups (e.g., halo C₁₋₆ alkyl), hydroxy alkyl groups (e.g., hydroxy C₁₋₆ alkyl), halo alkoxy groups (e.g., halo C₁₋₆ alkoxy), and the like. Further, the lipid component may contain fatty acid residues that are ester linked to one another.

The acidic glycolipid of the present disclosure may have the following general formula

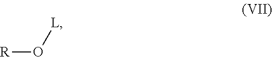

wherein L is a lipid group comprising a saturated or unsaturated fatty acid moiety or salt thereof containing from 6 to 30 carbon atoms (e.g., from 8 to 28 or from 10 to 26 carbon atoms), which may be linear or branched and optionally substituted, such as described in the preceding paragraph, and R is a carbohydrate group having from 1 to 5 monosaccharide residues, preferably from 1 to 3 (e.g., 1 or 2) monosaccharide residues, wherein each monosaccharide residue is independently a ring having 5 or 6 ring members, and one or more hydroxyl groups of the monosaccharide residues may be acylated (e.g., acetylated) or etherified.

The acidic glycolipids of the present disclosure may be produced, or obtained commercially, as known in the art, such as by extraction or fermentation processes of natural sources (e.g., natural or modified microorganisms, fungi, etc.) or by known chemical synthesis methods.

In many embodiments, the at least one acidic glycolipid of the present disclosure is a mixture of acidic glycolipids. In many embodiments, the at least one acidic glycolipid is present in a mixture with at least one non-acidic glycolipid (e.g., at least one lactonic glycolipid). Where the at least one acidic glycolipid is present in a mixture of acidic and non-acidic glycolipids, typically at least 25% by weight, for example, at least 50%, at least 60%, at least 70%, at least 80%, or at least 90% by weight, based on the total weight of glycolipids in the mixture are acidic glycolipids.

In many embodiments, the acidic glycolipid comprises at least one sophorolipid, at least one rhamnolipid or a mixture thereof. The at least one acidic glycolipid preferably comprises one or more sophorolipids, such as described herein.

Sophorolipids are generally composed of the disaccharide sophorose linked to fatty acids via a glycosidic bond. The fatty acid chain length generally varies, such as described herein for the lipid component (e.g., from 6 to 30, from 8 to 28 or from 10 to 26 carbon atoms) and may be saturated or unsaturated. Sophorolipids are generally known to exist in either of two general forms: acidic form or lactonic form. The acidic glycolipids of the present disclosure are preferably sophorolipids in acidic form (which can include salts thereof). The one or more acidic sophorolipids may be present in a mixture with one or more lactonic sophorolipids, such as described above.

In many embodiments, the at least one acidic glycolipid comprises at least one sophorolipid of formula (IV) or a salt thereof. In further embodiments, the at least one sophorolipid of formula (IV) is present in a mixture with at least one lactonic sophorolipid of formula (V)

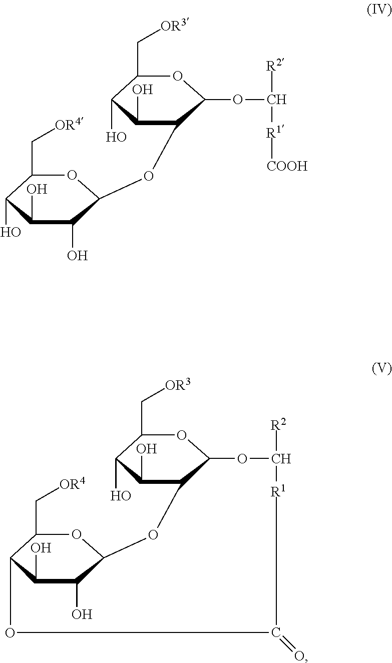

where R¹ and R^{1′} independently represent saturated hydrocarbon chains or single or multiple, in particular single, unsaturated hydrocarbon chains having from 8 to 20 carbon atoms, such as from 12 to 18 carbon atoms, in particular 14 to 18 or 14 to 16 carbon atoms, which can be linear or branched and can comprise one or more substituents, including, without limitation, halogen atoms, hydroxyl, lower (C₁₋₆) alkyl groups, halo lower (C₁₋₆) alkyl groups, hydroxy lower (C₁₋₆) alkyl groups, halo lower (C₁₋₆) alkoxy groups, and the like,
R² and R^{2′} independently represent a hydrogen atom or a saturated alkyl functional group or a single or multiple, in particular single, unsaturated alkyl functional group having from 1 to 9 carbon atoms, such as from 1 to 4 carbon atoms, which can be linear or branched and can comprise one or more hydroxy groups, and
R³, R^{3′}, R⁴ and R^{4′} independently represent a hydrogen atom or an acetyl group.

In many embodiments, R² and R^{2′} represent a methyl group or a hydrogen atom.

The sophorolipids may be produced, or obtained commercially, as known in the art, such as by fermentation processes of natural or modified microorganisms, for example, yeasts. Processes for the production of sophorolipids are described, for example, in WO2021183526, including techniques for adjusting the production, such as during fermentation, to increase the production of sophorolipids in acidic form relative to lactonic form. Sophorolipids in acidic form may be further isolated and purified by known techniques.

The acidic glycolipid may comprise at least one rhamnolipid. In general, rhamnolipids are composed of one or two rhamnose moieties (mono- or di-rhamnolipid) as the carbohydrate component linked to a fatty acid tail, often a 3-(hydroxyalkanoyloxy)alkanoic acid (HAA) fatty acid tail which may vary in length and degree of branching, such as, e.g., 3-hydroxydecanoic acid.

In many embodiments, the at least one rhamnolipid is of formula VI or a salt thereof

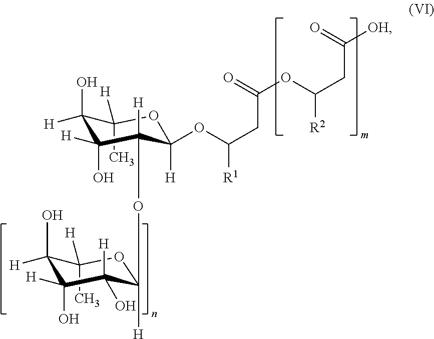

where m is 2, 1 or 0, n is 1 (dirhamnolipid) or 0 (monorhamnolipid), R¹ and R² are, independently of one another, the same or a different organic functional group having from 2 to 24 carbon atoms, such as from 5 to 13 carbon atoms, preferably a substituted (e.g., hydroxy-substituted) or unsubstituted, branched or unbranched alkyl functional group, which can also be unsaturated, wherein the alkyl functional group is preferably a linear saturated alkyl functional group having from 8 to 12 carbon atoms, such as a nonyl or a decyl functional group.

The rhamnolipids may be produced, or obtained commercially, as known in the art.

It shall be understood that the acidic glycolipids of the present disclosure may be provided at least partially in salt form. For example, the cations of the glycolipid salts may be chosen from Li⁺, Na⁺, K⁺, Mg²⁺, Ca²⁺, Al³⁺, NH₄⁺, primary ammonium ions, secondary ammonium ions, tertiary ammonium ions and quaternary ammonium ions.

The acidic glycolipids of the present disclosure may be used in a crude form from production or may be further processed such as by purification or isolation techniques.

In the compositions, formulations and methods of the present disclosure, the amount by weight of the at least one acidic glycolipid is greater than or about equal to the amount by weight of the at least one cationic antimicrobial. For example, the ratio by weight of the at least one acidic glycolipid to the at least one cationic antimicrobial may be at least 1.1:1, at least 1.2:1, at least 1.4:1, at least 1.6:1, at least 1.8:1, at least 2:1, at least 2.5:1, at least 3:1, at least 3.5:1, at least 4:1, at least 4.5:1 or at least 5:1. Typically, the ratio by weight of the at least one acidic glycolipid to the at least one cationic antimicrobial ranges from about 1:1, from 1.2:1, from 1.4:1, from 1.6:1, from 1.8:1, from 2:1, from 2.5:1, from 3:1, from 3.5:1, from 4:1, from 4.5:1 or from 5:1 to 100:1, to 75:1, to 50:1, to 40:1, to 35:1, to 30:1, to 25:1, to 20:1, to 15:1 or to 10:1. In many embodiments, the ratio by weight of the at least one acidic glycolipid to the at least one cationic antimicrobial ranges from about 1:1, from 1.2:1, from 1.4:1, from 1.6:1, from 1.8:1, from 2:1, from 2.5:1 or from 3:1 to 25:1, to 20:1, to 15:1 or to 10:1. In many embodiments, the ratio by weight of the at least one acidic glycolipid to the at least one cationic antimicrobial ranges from 1.2:1 to 20:1 or to 15:1, such as from 1.4:1 to 15:1, to 10.2:1 or to 10:1. The foregoing includes all ranges formed from any pair of any lower range limit and any upper range limit and including the endpoints thereof.

Acidic glycolipid content by weight can be determined according to techniques known in the art, preferably such as by HPLC or HPLC/MS.

The cationic antimicrobial and the acidic glycolipid are typically mixed in water and/or a water-miscible solvent to prepare the antimicrobial composition, and each individually may, but need not, be present in a solvent or suitable carrier at the time of combining. Suitable water-miscible solvents include, for example, glycerin, glycerol, glycols, polyethylene glycols, alcohols and the like. This mixture comprising the cationic antimicrobial and the acidic glycolipid may be used in or added to a wide variety of product formulations, as described further herein.

In general, the antimicrobial composition may comprise, for example, a combined amount of the at least one cationic antimicrobial (a) and the at least one acidic glycolipid (b) of from about 3 wt %, from about 4 wt %, from about 5 wt %, preferably from about 10 wt %, from about 15 wt %, or more preferably from about 20 wt % or from about 30 wt % to about 99 wt %, to about 95 wt %, preferably to about 90 wt %, more preferably to about 80 wt %, even more preferably to about 70 wt %, to about 60 wt % or to about 50 wt %, based on the weight of the antimicrobial composition. For example, the combined amount of the at least one cationic antimicrobial (a) and the at least one acidic glycolipid (b) may be from about 5 wt % or from about 10 wt % to about 95 wt %, preferably from about 10 wt % to about 90 wt %, or more preferably from about 20 wt % to about 80 wt % or from about 30 wt % to about 70 wt %. The composition may comprise, for example, (c) one or more solvents in an amount from about 1 wt %, from about 3 wt %, from about 5 wt %, preferably from about 10 wt % or more preferably from about 20 wt % or from about 30 wt % to about 97 wt %, to about 95 wt %, to about 90 wt %, to about 80 wt %, preferably to about 70 wt %, more preferably to about 60 wt %, even more preferably to about 50 wt % or to about 40 wt %, based on the weight of the antimicrobial composition. For example, the amount of component (c) may be from about 5 wt % to about 95 wt %, from about 10 wt % to about 90 wt %, from about 20 wt % to about 80 wt %, or from about 30 wt % to about 70 wt^{%}, preferably from about 10 wt % or from about 20 wt % to about 70 wt % or to about 60 wt %, more preferably from about 10 wt % or from about 20 wt % to about 60 wt %, to about 50 wt % or to about 40 wt %. The foregoing includes all ranges formed from any pair of any lower range limit and any upper range limit and including the endpoints thereof.

The antimicrobial composition may also contain one or more excipients (d), typically in minor quantities. Examples of excipients include, but are not limited to, a dispersant, solubilizer, buffer, stabilizer and/or preservative. When present, such one or more excipients typically collectively constitute from about 0.001 wt % to about 10 wt %, such as from about 0.01 wt %, from about 0.05 wt % or from about 0.1 wt % to about 5 wt %, to about 3 wt %, or to about 1 wt %, based on the weight of the antimicrobial composition. For example, the component (d) may range from 0 to 10 wt %, from about 0.01 wt % to about 5 wt %, from about 0.1 to about 5 wt %, from about 0.01 to about 3 wt %, from about 0.1 to about 3 wt %, from about 0.01 to about 1 wt %, or from about 0.1 to about 1 wt %. The foregoing includes all ranges formed from any pair of any lower range limit and any upper range limit and including the endpoints thereof.

The antimicrobial composition may also contain one or more non-acidic glycolipids (e), for example, lactonic glycolipids, in particular lactonic sophorolipids. Typically, the one or more non-acidic glycolipids are in a minority amount relative to the acidic glycolipids.

It shall be understood that the compositional percentages and ratios further relate to the presently disclosed methods for preparing the antimicrobial composition, that is, the components may be combined, for example, in accordance with the weight concentrations and ratios described herein.

In some embodiments, the antimicrobial composition consists essentially of at least one cationic antimicrobial (a), at least one acidic glycolipid (b), and at least one solvent (c), and optionally at least one excipient (d) and/or optionally at least one non-acidic glycolipid (e) (e.g., a lactonic sophorolipid).

In one aspect, the antimicrobial composition according to any embodiment herein does not contain a bacteriophage endolysin.

The present application provides for the use of the presently disclosed antimicrobial composition in product formulations, such as formulations for use as a personal care, home care, health care, food or industrial product. The present application also encompasses the use of the at least one cationic antimicrobial and the at least one acidic glycolipid in product formulations, such as formulations for use as a personal care, home care, health care, food or industrial product, where the at least one cationic antimicrobial and the at least one acidic glycolipid are added individually to the product formulation to provide the presently disclosed combination thereof. The at least one cationic antimicrobial and the at least one acidic glycolipid may be provided to the product formulation in the amounts and in the ratios by weight as described herein. Preferably, at least one or both of the at least one cationic antimicrobial and the at least one acidic glycolipid are present in a solvent or suitable carrier, such as described herein, for addition to the product formulation. It is believed that product formulations containing one or more anionic materials (i.e., an anionic formulation according to the present disclosure), such as containing at least one of an anionic rheology modifier, an anionic surfactant, or an anionic emulsifier, will benefit from the compatibilization of the cationic antimicrobial in accordance with the present disclosure even when the at least one cationic antimicrobial and the at least one acidic glycolipid are added individually to the product formulation. However, without wishing to be bound by any particular theory, it is believed that the benefits associated with the presently disclosed combination may be maximized by combining the cationic antimicrobial with the acidic glycolipid as described herein before applying the materials to an anionic product formulation. The anionic product formulation comprising the at least one cationic antimicrobial and the at least one acidic glycolipid is preferably free or substantially free of aggregates or precipitates.

In many embodiments, the anionic product formulation comprises, in addition to the antimicrobial composition, an anionic rheology modifier (e.g., anionic thickener) and/or an anionic surfactant (e.g., an anionic emulsifier).

Examples of suitable anionic thickeners include, without limitation, carboxylic acid polymers, crosslinked polyacrylate polymers, polyacrylamide polymers, polysaccharides, and gums.

Non-limiting examples of gums include acacia, agar, algin, alginic acid, ammonium alginate, amylopectin, calcium alginate, calcium carrageenan, carrageenan, dextrin, gelatin, gellan gum, karaya gum, konjac gum, locust bean gum, natto gum, potassium alginate, potassium carrageenan, propylene glycol alginate, sodium carboxymethyl dextran, sodium carrageenan, tragacanth gum, xanthan gum, and mixtures thereof.

Non-limiting examples of suitable polymeric anionic thickeners include homopolymers of acrylic acid with the INCI name Carbomer, copolymers of acrylic acid with alkyl acrylates with the INCI name Acrylates Copolymer, copolymers of acrylic acid with vinylpyrrolidone with the INCI name Acrylic Acid/VP Crosspolymer, copolymers of acrylic acid with C10-C30 alkyl acrylates with the INCI name Acrylates/C10-30 Alkyl Acrylates Crosspolymer, copolymers of acrylic acid with ethyl acrylate and associative alkyl acrylates with the INCI name Acrylates Copolymer or copolymers of (meth)acrylic acid with alkyl acrylates and ethoxylated hydrophobically modified alkyl acrylates with the INCI names Acrylates/Steareth-20 Methacrylate Copolymer, Acrylates/Beheneth-25 Methacrylate Copolymer, Acrylates/Steareth-20 Methacrylate Crosspolymer or the INCI Acrylates/Palmeth-25 Acrylates Copolymer. Polymeric anionic thickeners also include, without limitation, thickeners based on acrylamidomethylpropanesulfonic acid (AMPS), such as ammonium acryloyldimethyltaurate/VP copolymer or ammonium acryloyldimethyltaurate/beheneth-25 methacrylate copolymer.

The product formulation may contain, for example, at least one anionic thickener chosen from polyacrylic acid based polymers, xanthan gum, carbomers, carboxymethyl cellulose, and carrageenan.

Examples of suitable anionic surfactants or emulsifiers include, without limitation, those containing anionic functional groups at their head, such as sulfate, sulfonate, phosphate, and carboxylates.

Typical examples of anionic surfactants include, without limitation, soaps, alkyl benzenesulfonates, alkanesulfonates, olefin sulfonates, alkylether sulfonates, glycerol ether sulfonates, methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates and alkyl (ether) phosphates.

Examples of alkyl sulfates include ammonium lauryl sulfate, sodium lauryl sulfate (also called SDS, sodium dodecyl sulfate) and the related alkyl-ether sulfates, such as sodium lauryl ether sulfate (SLES), and sodium myreth sulfate. Carboxylates are common anionic surfactants and may comprise the alkyl carboxylates, such as sodium stearate.

In many embodiments, the product formulation contains an anionic surfactant or anionic emulsifier chosen from sulfates (e.g., sodium laureth or lauryl sulfate (SLES/SLS)), sulfonates, phosphates and carboxylates (e.g., alkyl sulfates, alkyl phosphates or alkyl carboxylates).

The antimicrobial composition of the present disclosure may also be used in non-ionic formulations and cationic formulations. The cationic antimicrobial and the acidic glycolipid may be added individually to the product formulation but are preferably added in the form of a mixture as described herein.

In general, an effective amount of the antimicrobial composition is added to a product formulation. Optimized amounts of the antimicrobial composition, and particular optimized ratios of the cationic antimicrobial and acidic glycolipid contained therein, may vary depending, for example, on the particular components selected, the end use application, the desired level of antimicrobial activity or preservation, the final properties of the product, and the like. As known in the art, an antimicrobial effect may be demonstrated by various suitable antimicrobial effectiveness tests (AET). Suitable test procedures and acceptance criteria are described, for example, in the U.S. Pharmacopeia, AET, the European Pharmacopeia (Efficacy of Antimicrobial Preservation), and the Japanese Pharmacopeia (Preservation Effectiveness Tests). Other suitable challenge tests may be used, such as those recommended by the FDA.

In general, the antimicrobial composition may be added to a product formulation, such as for use as a personal care, home care or other product, to provide, for example, a combined amount of the at least one cationic antimicrobial and the at least one acidic glycolipid of from about 0.01 wt %, from about 0.05 wt %, preferably from about 0.1 wt % or more preferably from about 0.2 wt % to about 3 wt %, to about 2.5 wt %, preferably to about 2 wt %, more preferably to about 1.5 wt % or even more preferably to about 1 wt %, based on the weight of the formulation. For example, the at least cationic antimicrobial and the at least one acidic glycolipid may be added to a product formulation to provide a combined amount of such materials of from about 0.01 to about 3 wt %, preferably from about 0.1 or 0.2 to about 2 wt % or more preferably from about 0.1 or 0.2 to about 1 wt %, based on the weight of the formulation. Preferably, the at least one cationic antimicrobial is added to the product formulation (and the formulation comprises the at least one cationic antimicrobial) in an amount no greater than 1 wt % or no greater than 0.75 wt %, more preferably in an amount no greater than 0.5 wt % or even more preferably in an amount no greater than 0.25 wt %, no greater than 0.2 wt % or no greater than 0.1 wt %. The foregoing ranges include all ranges formed from any pair of any lower range limit and any upper range limit and including the endpoints thereof.

The antimicrobial composition of the present disclosure is suitable for use in a wide range of product formulations and applications, such as for use in a personal care, home care, health care (such as pharmaceutical (i.e., having at least one active pharmaceutical ingredient), vitamin, nutraceutical or other health care products), food or industrial product. Examples include, without limitation, household products and cleaners, fabric detergents, dish detergents, cleansers, soaps, bubble baths, disinfectants, deodorizers, foods, food products, beverages, antimicrobial packaging, pharmaceutical products, medical devices, contact lenses, cosmetics, hygiene compositions, infant care products, antimicrobial soaps, hand sanitizers, deodorants, antiperspirants, anti-microbial coatings, dental compositions, toothpastes, mouthwashes, lipsticks, dental appliances, medications, athlete's foot treatments, medicated chewing gums, wound care compositions, dermatological compositions, acne treatments, skin conditioners, skin moisturizers, anti-wrinkle formulations, skin whiteners, sunscreens, tanning lotions, hair products, shampoos, shower gels, bubble baths, conditioners, shaving creams, spermicides etc. Also included are microbial-resistant fabrics and apparel, anti-microbial condoms, surgical gowns, microbial-resistant hospital equipment, anti-microbial paper products, animal care products, antimicrobial plastics, antimicrobial plastic devices, rubbers and other fabrication materials, appliances with antimicrobial constituents or coatings, etc.

In general, the product formulation comprises the antimicrobial composition of the present disclosure and one or more additional ingredients suitable for use in a personal care (e.g., cosmetic), home care, health care, or industrial product formulation or food product.

As a particular example, the antimicrobial composition can be incorporated into any number and variety of different personal care products, such as cosmetic products. For example, the antimicrobial composition of the present disclosure may be used in personal care products used for skin care, hair care, oral care, cosmetics, personal cleaning, hygiene, sun protection and other personal care applications. Examples of such products include, without limitation, skin toners, skin cleansers, night creams, skin creams, shaving creams, skin lotions, makeup, mascara, lipstick, blush, gloss, eye-liner, makeup removers, sunscreens, lip balms, fragrances, massage oils, shampoos, conditioners, hair styling gels, hair reparatives, hair tonics, hair fixatives, hair mousses, bath and shower gels, liquid soaps, moisturizing sprays, bath additives, ophthalmic preparations, foaming soaps and body washes, liquids for any personal care wet wipe application, etc.

As a further example, the antimicrobial composition can be incorporated into any number and variety of different home care products, such as fabric care products and cleaning products. Examples include dish soaps, laundry detergents, cleaning wipes, cleaning formulations and other home care applications.

Depending on the application, the product formulation may contain numerous and different compatible ingredients. A personal care formulation may contain, for example, any of solvents, surfactants, emulsifiers, chelating agents, oxidizing agents, colorants, rheology modifiers, conditioners, emollients, skin care ingredients, moisturizers, thickeners, humectants, fillers, antioxidants, other antimicrobial agents or preservatives, active ingredients, such as dermatologically active ingredients typically suited for topical application, fragrances, etc.

A home care product formulation may contain, for example, any of cleaning agents, detergents, emulsifiers, surfactants, thickeners, gelling agents, bleaches, whiteners, deodorizers, enzymes, stabilizers, fragrances, soil-release agents, anti-shrinking agents, anti-wrinkle agents, anti-spotting agents, antioxidants, UV absorbing compounds, anti-corrosion agents, anti-static agents, ironing aids, odor-preventing compounds, etc.

Examples of the foregoing ingredients and other agents used in personal care and home care product compositions are known in the art.

The antimicrobial composition may be included in product formulations of a variety of different forms, for example, liquids, pastes, serums, hydrogels, creams, emulsions, lotions, gels, oils, wipes, ointments, semi-solid compositions, foams and aerosol sprays. The antimicrobial composition may be used in a leave-on or rinse-off product formulation. The antimicrobial composition can be used in hair care products, such as shampoos, hair conditioners, hair dyes, hair tonic, hair gel, hair dressings, hair grooming aids and other hair care preparations; shaving applications such as shaving cream, aftershave lotions, and other shaving applications; personal cleaners for the body and hands, such as liquid bath soaps and detergents; fragrance preparations, such as perfumes, after bath splashes, and other similar fragrant preparations, skin care products, such as moisturizers, creams, and lotions and other similar skin care products, make-up products, such as mascara, base foundations and the like; make-up removal products, sun care products, indoor tanning products and other similar personal care products. The antimicrobial composition may be incorporated in formulations used to saturate wipes used for personal cleaning and hygiene, for example baby wipes, wet toilet wipes, make-up removal wipes and exfoliating wipes the like, or wet wipe formulations for home care.

Preferably, when added to a product formulation, the antimicrobial composition provides an antimicrobial effect according to the challenge test procedures in the below Examples achieving a passing growth rating according to the criteria associated with Table 3 herein.

The antimicrobial composition of the present disclosure may be used in product formulations containing other antimicrobial, preservative or booster agents. Examples of such agents include those suitable for use in various industries in which the antimicrobial composition of the present disclosure is beneficial. Examples of suitable additional antimicrobial or booster agents include, but are not limited to, 1,2-decanediol, 1,2-hexanediol, 1,3-propanediol, 4-hydroxyacetophenone, amyl cinnamic aldehyde, benzyl alcohol, caprylhydroxamic acid, caprylyl glycol, cinnamic alcohol, cinnamic aldehyde, ethylhexylglycerin, gluconolactone, glyceryl caprate/caprylate, phenethyl alcohol, phenoxyethanol, potassium benzoate, propylene glycol caprylate, p-thymol, salicylic acid, sodium anisate, sodium benzoate, sodium dehydroacetate, sodium levulinate, sorbic acid, sorbitan caprylate, and combinations thereof. The at least one additional antimicrobial or booster agent may be present in an amount typically no greater than 1 wt %, preferably no greater than 0.75 wt % or no greater than 0.5 wt %, more preferably in an amount of less than 0.4 wt % or less than 0.3 wt %, such as in an amount of from about 0.05 wt % to less than 0.3 wt % (e.g., from about 0.05 wt % to about 0.25 wt %).

Preferably, the at least one additional antimicrobial or booster agent is chosen from sodium benzoate, glyceryl caprylate, 4-hydroxyacetophenone, sodium dehydroacetate, sodium anistate, 1,2-hexanediol, gluconolactone, benzyl alcohol, and combinations thereof.

The present disclosure further includes a method of controlling or inhibiting microbial growth in or preserving a product formulation, such as a formulation for use as a personal care, home care, health care, food or industrial product. The method comprises adding an effective amount of at least one cationic antimicrobial and at least one acidic glycolipid to the product formulation, wherein the amount by weight of the at least one acidic glycolipid is greater than or about equal to the amount by weight of the at least one cationic antimicrobial, such as at the ratios by weight described herein. Preferably, the product formulation is an anionic formulation. The at least one cationic antimicrobial and at least one acidic glycolipid may be added to the product formulation individually or in the form of a mixture, preferably in the form of a mixture, such as described herein for the antimicrobial composition, especially for addition to an anionic product formulation. The method may further include preparing the antimicrobial composition by combining the at least one cationic antimicrobial and the at least one acidic glycolipid, typically in water and/or a water-miscible solvent, prior to adding the at least one cationic antimicrobial and the at least one acidic glycolipid to a product formulation, especially an anionic formulation.

Also provided is a method of preparing an antimicrobial composition comprising combining at least one cationic antimicrobial and at least one acidic glycolipid, wherein the amount by weight of the at least one acidic glycolipid is greater than or about equal to the amount by weight of the at least one cationic antimicrobial, such as at the ratios by weight described herein. Preferably, the at least one cationic antimicrobial and the at least one acidic glycolipid are combined in water and/or a water-miscible solvent. Preferably, the at least one cationic antimicrobial and the at least one acidic glycolipid are combined prior to use in or addition to a product formulation, especially an anionic product formulation.

Also provided is a method of preparing or formulating at least one cationic antimicrobial for use in an anionic product formulation, the method comprising combining the at least one cationic antimicrobial with an acidic glycolipid, typically in water and/or a water-miscible solvent, prior to use or addition of the cationic antimicrobial in an anionic product formulation, wherein the amount by weight of the at least one acidic glycolipid is greater than or about equal to the amount by weight of the at least one cationic antimicrobial, such as at the ratios by weight described herein.

Further provided is a method of preparing or formulating a product formulation, such as for use as a personal care, home care, health care, food, or industrial product, comprising adding an effective amount of at least one cationic antimicrobial and at least one acidic glycolipid to the product formulation, wherein the amount by weight of the at least one acidic glycolipid is greater than or about equal to the amount by weight of the at least one cationic antimicrobial, such as at the ratios by weight described herein. The at least one cationic antimicrobial and the at least one acidic glycolipid may be added to the product formulation individually or in the form of a mixture, preferably in the form of a mixture, such as described herein for the antimicrobial composition, especially for addition to anionic product formulations. Preferably, the product formulation is an anionic formulation.

The present disclosure further includes, without limitation, the following exemplary embodiments:

Embodiment 1: A product formulation comprising at least one of an anionic rheology modifier, an anionic surfactant or an anionic emulsifier; and an antimicrobial effective amount of a combination of at least one cationic antimicrobial and at least one acidic glycolipid, wherein the cationic antimicrobial comprises or is
(i) a compound having one or more cationic guanidine or amidine functional groups; or
(ii) a N^{α}-alkanoyl dibasic amino acid ester salt,
   the at least one acidic glycolipid comprises or is at least one acidic sophorolipid, and
   the amount by weight of the at least one acidic glycolipid is greater than or about equal to the amount by weight of the at least one cationic antimicrobial.

Embodiment 2: The product formulation according to Embodiment 1, wherein the cationic antimicrobial comprises or is an alkyl guanidine, a biguanide, a polybiguanide, a salt thereof or a combination thereof.

Embodiment 3: The product formulation according to Embodiment 1, wherein the cationic antimicrobial comprises or is dodecylguanidine, chlorhexidine, alexidine, polyhexamethylene biguanide, polyaminopropyl biguanide, hexamidine, a salt thereof or a combination thereof.

Embodiment 4: The product formulation according to Embodiment 1, wherein the cationic antimicrobial comprises or is a N^{α}—(C₆-C₂₀)alkanoyl dibasic amino acid (C₁-C₆)alkyl ester salt.

Embodiment 5: The product formulation according to Embodiment 1, wherein the cationic antimicrobial comprises or is a N^{α}-alkanoyl dibasic amino acid ester salt of the formula (I)

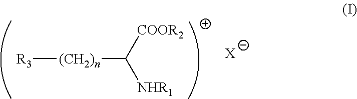

where R₁ is a straight alkyl chain from a saturated fatty acid or a hydroxy acid having from 6 to 20 carbon atoms bonded to the α-amino group through an amidic bond,
R₂ is a straight or branched alkyl chain from 1 to 6 carbon atoms, or an aromatic group,
R₃ is selected from

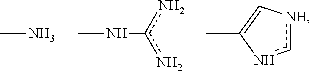

n is from 1 to 6, and
X⁻ is Cl⁻, Br⁻ or a counter ion derived from an organic or inorganic acid or a phenolic compound.

Embodiment 6: The product formulation according to Embodiment 1, wherein the cationic antimicrobial comprises or is a N^{α}—(C₆-C₂₀)alkanoyl-L-arginine (C₁-C₆)alkyl ester salt.

Embodiment 7: The product formulation according to Embodiment 1, wherein the cationic antimicrobial comprises or is ethyl lauroyl arginate HCl (LAE), pyrrolidone carboxylic acid (PCA) ethyl cocoyl arginate, chlorhexidine digluconate or a combination thereof.

Embodiment 8: The product formulation according to any preceding Embodiment, wherein the at least one acidic sophorolipid is of the formula (IV)

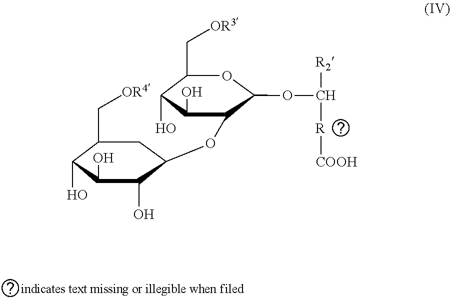

where R^{1′} is an optionally substituted saturated or unsaturated hydrocarbon chain having 8 to 20 carbon atoms, which can be linear or branched;
R^{2′} is a hydrogen atom or a saturated or unsaturated alkyl functional group having from 1 to 9 carbon, which can be linear or branched, and is optionally substituted with one or more hydroxy groups; and
R^{3′} and R^{4′} are independently a hydrogen atom or an acetyl group.

Embodiment 9: The product formulation according to any preceding Embodiment, wherein the ratio by weight of the acidic glycolipid to the cationic antimicrobial is from about 1:1 to 100:1, preferably wherein the ratio is from 1.2:1 to 20:1.

Embodiment 10: The product formulation according to any preceding Embodiment, wherein the product formulation comprises at least one anionic rheology modifier comprising a carboxylic acid polymer, a crosslinked polyacrylate polymer, a polyacrylamide polymer, a polysaccharide, a gum or a combination thereof.

Embodiment 11: The product formulation according to Embodiment 10, wherein the anionic rheology modifier comprises a polyacrylic acid based polymer, xanthan gum, a carbomer, carboxymethyl cellulose, a carrageenan or a combination thereof.

Embodiment 12: The product formulation according to any preceding Embodiment, wherein the product formulation comprises at least one anionic surfactant comprising a sulfate, a sulfonate, a phosphate, a carboxylate or a combination thereof.

Embodiment 13: The product formulation according to any preceding Embodiment, wherein the product formulation is a personal care or home care product formulation.

Embodiment 14: The product formulation according to any preceding Embodiment, wherein the at least one cationic antimicrobial is present in an amount no greater than 0.5 wt %, preferably in an amount no greater than 0.25 wt %, based on the weight of the formulation.

Embodiment 15: The product formulation according to any preceding Embodiment, wherein the formulation further comprises at least one additional antimicrobial or booster agent chosen from 1,2-decanediol, 1,2-hexanediol, 1,3-propanediol, 4-hydroxyacetophenone, amyl cinnamic aldehyde, benzyl alcohol, caprylhydroxamic acid, caprylyl glycol, cinnamic alcohol, cinnamic aldehyde, ethylhexylglycerin, gluconolactone, glyceryl caprate, glyceryl caprylate, phenethyl alcohol, phenoxyethanol, potassium benzoate, propylene glycol caprylate, p-thymol, salicylic acid, sodium anisate, sodium benzoate, sodium dehydroacetate, sodium levulinate, sorbic acid, sorbitan caprylate, and combinations thereof.

Embodiment 16: The product formulation of Embodiment 15, wherein the at least one additional antimicrobial or booster agent is present in an amount no greater than 0.5 wt %, preferably in an amount of less than 0.3 wt %, based on the weight of the formulation.

Embodiment 17: The product formulation of Embodiment 15 or 16, wherein the at least one additional antimicrobial or booster agent is chosen from sodium benzoate, glyceryl caprylate, 4-hydroxyacetophenone, sodium dehydroacetate, sodium anistate, 1,2-hexanediol, gluconolactone, benzyl alcohol, and combinations thereof.

Embodiment 18: A method of preparing the product formulation according to any one of Embodiments 1-17, comprising providing a formulation comprising at least one of an anionic rheology modifier, an anionic surfactant or an anionic emulsifier, and adding an antimicrobial effective amount of the at least one acidic glycolipid and the at least one cationic antimicrobial to the formulation.

Embodiment 19: The method of Embodiment 18, wherein the at least one cationic antimicrobial and the at least one acidic glycolipid are added to the formulation in the form of a mixture.

Embodiment 20: A method of controlling microbial growth in or preserving a product formulation comprising at least one of an anionic rheology modifier, an anionic surfactant or an anionic emulsifier, the method comprising adding to the product formulation an antimicrobial effective amount of at least one cationic antimicrobial and at least one acidic glycolipid, wherein the cationic antimicrobial comprises or is
(i) a compound having one or more cationic guanidine or amidine functional groups; or
(ii) a N^{α}-alkanoyl dibasic amino acid ester salt,
   the at least one acidic glycolipid comprises or is at least one acidic sophorolipid, and the amount by weight of the at least one acidic glycolipid is greater than or about equal to the amount by weight of the at least one cationic antimicrobial.

Embodiment 21: The method of Embodiment 20, wherein the at least one cationic antimicrobial and the at least one acidic glycolipid are added to the product formulation in the form of a mixture.

Embodiment 22: The method of Embodiment 20 or 21, wherein the cationic antimicrobial comprises or is an alkyl guanidine, a biguanide, a polybiguanide, a salt thereof or a combination thereof.

Embodiment 23: The method of Embodiment 20 or 21, wherein the cationic antimicrobial comprises or is dodecylguanidine, chlorhexidine, alexidine, polyhexamethylene biguanide, polyaminopropyl biguanide, hexamidine, a salt thereof or a combination thereof.

Embodiment 24: The method of Embodiment 20 or 21, wherein the cationic antimicrobial comprises or is a N^{α}—(C₆-C₂₀)alkanoyl dibasic amino acid (C₁-C₆)alkyl ester salt.

Embodiment 25: The method of Embodiment 20 or 21, wherein the cationic antimicrobial comprises or is a N^{α}-alkanoyl dibasic amino acid ester salt of the formula (I)

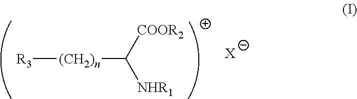

where R₁ is a straight alkyl chain from a saturated fatty acid or a hydroxy acid having from 6 to 20 carbon atoms bonded to the α-amino group through an amidic bond,
R₂ is a straight or branched alkyl chain from 1 to 6 carbon atoms, or an aromatic group,
R₃ is selected from

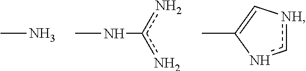

n is from 1 to 6, and
X⁻ is Cl⁻, Br⁻ or a counter ion derived from an organic or inorganic acid or a phenolic compound.

Embodiment 26: The method of Embodiment 20 or 21, wherein the cationic antimicrobial comprises or is a N^{α}—(C₆-C₂₀)alkanoyl-L-arginine (C₁-C₆)alkyl ester salt.

Embodiment 27: The method of Embodiment 20 or 21, wherein the cationic antimicrobial comprises or is ethyl lauroyl arginate HCl (LAE), pyrrolidone carboxylic acid (PCA) ethyl cocoyl arginate, chlorhexidine digluconate or a combination thereof.

Embodiment 28: The method of any one of Embodiments 20-27, wherein the at least one acidic sophorolipid is of the formula (IV)

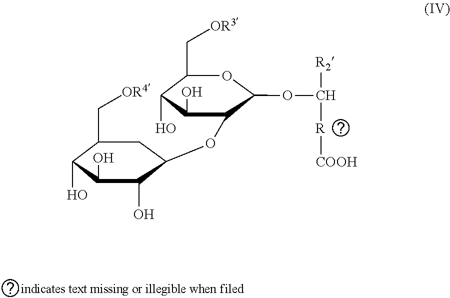

where R^{1′} is an optionally substituted saturated or unsaturated hydrocarbon chain having 8 to 20 carbon atoms, which can be linear or branched;
R^{2′} is a hydrogen atom or a saturated or unsaturated alkyl functional group having from 1 to 9 carbon, which can be linear or branched, and is optionally substituted with one or more hydroxy groups; and
R^{3′} and R^{4′} are independently a hydrogen atom or an acetyl group.

Embodiment 29: The method according to any one of Embodiments 20-28, wherein the at least one acidic sophorolipid is present in a mixture with at least one lactonic sophorolipid, wherein at least 50% by weight, such as at least 60%, at least 70%, at least 80%, or at least 90% by weight, based on the total weight of sophorolipids in the mixture, are in acidic form.

Embodiment 30: The method according to Embodiment 29, wherein formula (IV) represents the at least one acidic sophorolipid and formula (V) represents the at least one lactonic sophorolipid:

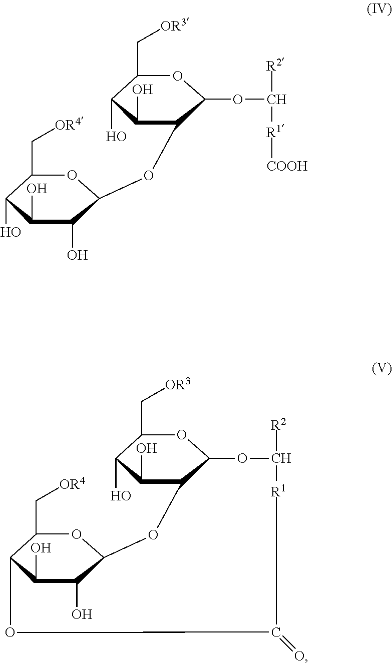

where R¹ and R^{1′} are each independently an optionally substituted saturated or unsaturated hydrocarbon chain having 8 to 20 carbon atoms, which can be linear or branched;
R² and R^{2′} are each independently a hydrogen atom or a saturated or unsaturated alkyl functional group having from 1 to 9 carbon, which can be linear or branched, and is optionally substituted with one or more hydroxy groups; and
R³, R^{3′}, R⁴ and R^{4′} are each independently a hydrogen atom or an acetyl group.

Embodiment 31: The method of any one of Embodiments 20-30, wherein the ratio by weight of the acidic glycolipid to the cationic antimicrobial is from about 1:1 to 100:1, preferably wherein the ratio is from 1.2:1 to 20:1.

Embodiment 32: The method of any one of Embodiments 20-31, wherein the product formulation is a personal care or home care product formulation.

Embodiment 33: The method of any one of Embodiments 20-32, wherein the at least one cationic antimicrobial is added in an amount no greater than 0.5 wt %, preferably no greater than 0.25 wt %, based on the weight of the formulation.

Embodiment 34: The method of any one of Embodiments 20-33, wherein the method further comprises adding to the formulation at least one additional antimicrobial or booster agent chosen from 1,2-decanediol, 1,2-hexanediol, 1,3-propanediol, 4-hydroxyacetophenone, amyl cinnamic aldehyde, benzyl alcohol, caprylhydroxamic acid, caprylyl glycol, cinnamic alcohol, cinnamic aldehyde, ethylhexylglycerin, gluconolactone, glyceryl caprate, glyceryl caprylate, phenethyl alcohol, phenoxyethanol, potassium benzoate, propylene glycol caprylate, p-thymol, salicylic acid, sodium anisate, sodium benzoate, sodium dehydroacetate, sodium levulinate, sorbic acid, sorbitan caprylate, and combinations thereof.

Embodiment 35: The method of Embodiment 34, wherein the at least one additional antimicrobial or booster agent is added in an amount no greater than 0.5 wt %, preferably in an amount less than 0.3 wt %, based on the weight of the formulation.

Embodiment 36: The method of Embodiment 34 or 35, wherein the at least one additional antimicrobial or booster agent is chosen from sodium benzoate, glyceryl caprylate, 4-hydroxyacetophenone, sodium dehydroacetate, sodium anistate, 1,2-hexanediol, gluconolactone, benzyl alcohol, and combinations thereof.

Embodiment 37: An antimicrobial composition comprising from about 10 wt % to about 95 wt %, preferably from about 10 wt % to about 90 wt %, more preferably from about 20 wt % to about 80 wt %, even more preferably from about 30 wt % to about 70 wt %, based on the total weight of the antimicrobial composition, of a combination of (a) at least one cationic antimicrobial and (b) at least one acidic glycolipid, wherein the cationic antimicrobial comprises or is
(i) a compound having one or more cationic guanidine or amidine functional groups; or
(ii) a N^{α}-alkanoyl dibasic amino acid ester salt, and
   the at least one acidic glycolipid comprises or is at least one acidic sophorolipid, and
   the ratio by weight of (b) to (a) is from about 1:1 to 100:1.

Embodiment 38: The antimicrobial composition according to Embodiment 37, wherein the cationic antimicrobial comprises or is an alkyl guanidine, a biguanide, a polybiguanide, a salt thereof or a combination thereof.

Embodiment 39: The antimicrobial composition according to Embodiment 37, wherein the cationic antimicrobial comprises or is dodecylguanidine, chlorhexidine, alexidine, polyhexamethylene biguanide, polyaminopropyl biguanide, hexamidine, a salt thereof or a combination thereof.

Embodiment 40: The antimicrobial composition according to Embodiment 37, wherein the cationic antimicrobial comprises or is a N^{α}—(C₆-C₂₀)alkanoyl dibasic amino acid (C₁-C₆)alkyl ester salt.

Embodiment 41: The antimicrobial composition according to Embodiment 37, wherein the cationic antimicrobial comprises or is a N^{α}-alkanoyl dibasic amino acid ester salt of the formula (I)

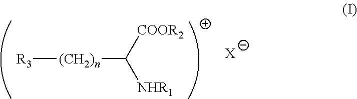

where R₁ is a straight alkyl chain from a saturated fatty acid or a hydroxy acid having from 6 to 20 carbon atoms bonded to the α-amino group through an amidic bond,
R₂ is a straight or branched alkyl chain from 1 to 6 carbon atoms, or an aromatic group,
R₃ is selected from

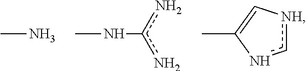

n is from 1 to 6, and
X⁻ is Cl⁻, Br⁻ or a counter ion derived from an organic or inorganic acid or a phenolic compound.

Embodiment 42: The antimicrobial composition according to Embodiment 37, wherein the cationic antimicrobial comprises or is a N^{α}—(C₆-C_{2M})alkanoyl-L-arginine (C₁-C₆)alkyl ester salt.

Embodiment 43: The antimicrobial composition according to Embodiment 37, wherein the cationic antimicrobial comprises or is ethyl lauroyl arginate HCl (LAE), pyrrolidone carboxylic acid (PCA) ethyl cocoyl arginate, chlorhexidine digluconate or a combination thereof.

Embodiment 44: The antimicrobial composition according to any one of Embodiments 37-43, wherein the at least one acidic sophorolipid is of the formula (IV)

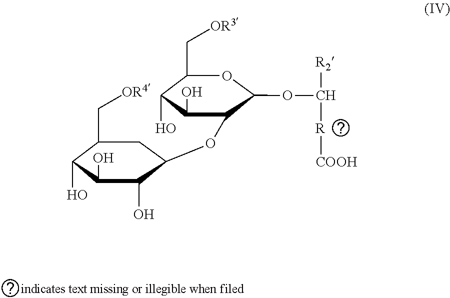

where R^{1′} is an optionally substituted saturated or unsaturated hydrocarbon chain having 8 to 20 carbon atoms, which can be linear or branched;
R^{2′} is a hydrogen atom or a saturated or unsaturated alkyl functional group having from 1 to 9 carbon, which can be linear or branched, and is optionally substituted with one or more hydroxy groups; and
R^{3′} and R^{4′} are independently a hydrogen atom or an acetyl group.

Embodiment 45: The antimicrobial composition according to any one of Embodiments 37-44, wherein the ratio by weight of the acidic glycolipid to the cationic antimicrobial is from 1.2:1 to 20:1.

Embodiment 46: The antimicrobial composition according to any one of Embodiments 37-45, wherein the combined amount of (a) and (b) is from about 20 wt % to about 80 wt %, based on the total weight of the antimicrobial composition.

As used herein, the articles “a”, “an”, and “the” preceding an element or component of the invention are intended to be nonrestrictive regarding the number of instances (i.e., occurrences) of the element or component. Therefore “a”, “an”, and “the” should be read to include one or at least one, and the singular word form of the element or component also includes the plural unless the number is obviously meant to be singular.

As used herein, the term “comprising” means the presence of the stated features, integers, steps, or components as referred to in the claims, but that it does not preclude the presence or addition of one or more other features, integers, steps, components or groups thereof. The term “comprising” is intended to include embodiments encompassed by the terms “consisting essentially of” and “consisting of,” unless the context dictates otherwise.

As used herein, the term “about” modifying the quantity of an ingredient employed refers to variation in the numerical quantity that can occur, for example, through typical measuring and handling procedures used for making concentrates or use solutions in the real world; through inadvertent error in these procedures; through differences in the manufacture, source, or purity of the ingredients employed to make the compositions or carry out the methods; and the like.

Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like.

When a parameter is given either as a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range.

### EXAMPLES

### Challenge Testing Procedure

Preservative activity in representative formulations was evaluated by using a modification of the Personal Care Products Council (PCPC) protocol (Machtiger, et al. 2001. Determination of the Efficacy of Preservation of Non-Eye Area Water Miscible Cosmetic and Toiletry Formulations: Collaborative Study. J of AOAC International. 84:1, 101-109). The evaluation was conducted using a four week, two-cycle microbial preservative efficacy test (challenge test) at 25° C. to determine the preservative efficacy of each treatment.

Table 1 shows an exemplary formulation for xanthan gum based anionic lotion compositions. Xanthan gum is commonly used as a natural rheological modifier in personal care products. The formulation below was made by heating the aqueous (A) and oil (B) phases separately to 75-80° C. The oil phase was then poured into the aqueous phase and mixed by an overhead mixer and a homogenizer. The oil-in-water emulsion was cooled to room temperature. Sodium hydroxide solution was used to adjust the pH.

TABLE 1
Representative lotion anionic
formulation with xanthan gum

| Phase | Ingredients | WT % |
|---|---|---|
| A | Water | 79.70 |
| | Xanthan Gum | 0.30 |
| | Propylene Glycol | 4.00 |
| B | Cetearyl Alcohol and | 2.00 |
| | Ceteareth-20 | |
| | Stearic Acid | 3.00 |
| | Glyceryl Stearate | 2.00 |
| | Petrolatum | 2.00 |
| | Ethylhexyl Olivate | 4.00 |
| | Isopropyl Myristate | 3.00 |

Lotion formulation samples were dosed with certain test materials as shown further below and mixed thoroughly at room temperature, and the formulation samples were divided into two separate aliquots of 5 gram each. One aliquot was inoculated with 50 μl of a diluted bacterial pool, the second aliquot was inoculated with 50 μl of a diluted fungal pool as described below. Unpreserved product samples were included in the test as a growth control.

A mixed bacterial inoculum was prepared using 24-hour cultures of the test bacteria (Table 2) grown in trypticase soy broth (TSB). Equal volumes of the bacterial test strains were combined and diluted one to ten in phosphate buffer to obtain an inoculum of approximately 5×10⁷ to 5×10⁸ colony forming units per ml (cfu/ml). The test samples were inoculated with 1% of the mixed bacterial inoculum. A mixed fungal inoculum was prepared using cell suspensions of the yeast Candida albicans ATCC #10231 and the mold Aspergillus brasiliensis ATCC #16404 in phosphate buffer. Equal volumes of the fungal test strains were combined and diluted one to ten with phosphate buffer to obtain an inoculum of approximately 5×10⁶ to 5×10⁷ cfu/ml. The test samples were inoculated with 100 of the mixed fungal inoculum.

TABLE 2
Mixed Bacterial Inoculum

| Microorganism | ATCC Number | Type |
|---|---|---|
| Burkholderia cepacia | 25416 | Gram-Negative |
| | | Non-fermenter |
| Enterobacter gergoviae | 33028 | Gram-Negative |
| | | Fermenter |
| Escherichia coli | 8739 | Gram-Negative |
| | | Fermenter |
| Klebsiella pneumoniae | 13883 | Gram-Negative |
| | | Fermenter |
| Pseudomonas aeruginosa | 9027 | Gram-Negative |
| | | Non-fermenter |
| Pseudomonas aeruginosa | 15442 | Gram-Negative |
| | | Non-fermenter |
| Pseudomonas putida | 49128 | Gram-Negative |
| | | Non-fermenter |
| Staphylococcus aureus | 6538 | Gram-Positive |
| Staphylococcus epidermidis | 12228 | Gram-Positive |

The samples were subjected to microbial challenge at time zero and inoculated a second time after seven days. The number of microorganisms added to each sample was determined by a standard Most Probable Number (MPN) determination in Trypticase Soy Broth (TSB) for bacteria and Potato Dextrose Broth (PDB) for fungi. Samples were incubated at 25° C. for the test duration of four weeks. Samples were monitored for bacterial and fungal contamination after 2, 7, 14, 21, and 28 days. Samples challenged with bacteria were streak-plated onto Trypticase Soy Agar (TSA) and incubated at 30° C. for 24 hours. Samples challenged with fungi were streak-plated onto Potato Dextrose Agar (PDA) and incubated at 25° C. for 7 days. After incubation, plates were given a Growth Rating to determine the colony forming units per gram (CFU/g) present in each test sample at the testing time point. Table 3 represents the growth score. As is used by various challenge test methods, the passing criteria is more stringent for bacteria than for fungi. A rating of 2, 3, or 4 at 21 and/or 28 days was considered a failure for bacteria. For fungi, a rating of 2 at 21 and/or 28 days was considered a pass.

TABLE 3
Growth score used to determine CFU/mL in test samples

| Growth | Description of | | Approximate |
|---|---|---|---|
| Rating | Microbial Growth | Plating Results | cfu/g |
| 0 | No | No detectable | <10 |
| | contamination | growth | |
| 1 | Trace | 1 to 9 | 1 × 10¹ to 9 × 10¹ |
| | contamination | colonies | |
| 2 | Light | 10 to 99 | 1 × 10² to 9.9 × 10² |
| | contamination | colonies | |
| 3 | Moderate | 100 to 1,000 | 1 × 10³ to 9.9 × 10³ |
| | contamination | colonies | |
| 4 | Heavy | >1,000 or | >1 × 10⁴ |
| | contamination | smear | |

For bacteria challenge testing, a composition was scored as “Pass” if the composition obtained a growth rating score caused by bacteria of 1 or less at days 21-28 of the testing period and was scored as “Fail” if the composition obtained a growth rating score caused by bacteria of 2 or more at days 21-28 of the testing period.

For fungi challenge testing, a composition was scored as “Pass” if the composition obtained a growth rating score caused by fungi of 2 or less at days 21-28 of the testing period and was scored as “Fail” if the composition obtained a growth rating score caused by fungi of more than 2 at days 21-28 of the testing period.

The “Pass” rating of a dosed composition was also contingent upon the unpreserved sample having a rating of “Fail”.

### Test Materials and Abbreviations

CHD.G: Chlorhexidine digluconate
GC: Glyceryl caprylate
GL-A: Sophorolipid mixture in water (approximately 90:10 weight ratio of acidic to lactonic sophorolipids of Formulas (TV) and (V) herein, respectively)
GL-L: Sophorolipid mixture in water (approximately 30:70 weight ratio of acidic to lactonic sophorolipids of Formulas (IV) and (V) herein, respectively)
HAP: 4-Hydroxyacetophenone
LAE: Ethyl lauroyl arginate HCl
NaBz: Sodium benzoate
XG: Xanthan gum lotion

The ethyl lauroyl arginate HCl (LAE) used when forming a mixture with the acidic glycolipid was a solid material of more than 85% purity and was a 20% wt solution of LAE in glycerin when dosed alone (as a control) into the test formulation.

The chlorhexidine digluconate used in the testing was a 20 wt % solution in water.

Solutions comprising LAE and glycolipids were prepared by dissolving the high purity solid LAE material into the CL-A or GL-L glycolipid mixtures. Such mixtures were prepared by using about 1% to 20% LAE in the glycolipids/water mixtures. Solutions comprising CHD.G and glycolipids were prepared using about 5% CHD.G in the GL-A glycolipid mixture. The resulting solutions were dosed as test samples into a representative lotion formulation.

In particular, a representative lotion formulation with xanthan gum was made according to the composition described in Table 1. This unpreserved anionic xanthan gum lotion was dosed with various test samples at room temperature and thoroughly mixed, according to the samples and weight percentages described in Tables 4 and 5. The concentration of LAE and CHD.G in the sample formulations is shown by ppm. The percentage by weight of acidic glycolipids in the sample formulations and the weight ratio of acidic glycolipids to LAE or CHD.G are shown in the third and fourth columns, respectively.

As shown, none of the lotion formulations passed the challenge test when dosed with a solution of LAE, CHD.G, GL-A, or GL-L alone or with a mixture of LAE and GL-L where the weight ratio of acidic glycolipids to LAE was approximately 0.5. However, when the anionic formulations were dosed with the mixtures of LAE or CHD.G and GL-A where the ratio of acidic glycolipids to LAE or CHD.G were as shown for Samples I through L and N, the mixtures provided effective antimicrobial activity and passed the challenge criteria as shown in Table 4.

TABLE 4

| | | | Acidic | | | | | |
|---|---|---|---|---|---|---|---|---|
| | | Acidic | Glycolipids | Bacteria | | Fungi | | |
| | Preservative | Glycolipids | to /LAE or | D | D | D | D | Overall |
| Sample | Treatment | Conc. [% wt] | CHD.G Ratio | 21 | 28 | 21 | 28 | Result |
| Comparative | Xanthan Gum | | | 4 | 4 | 4 | 4 | FAIL |
| A | Lotion (XG) | | | | | | | |
| | blank | | | | | | | |
| Comparative | XG/LAE 1000 | | | 4 | 4 | 2 | 2 | FAIL |
| B | ppm | | | | | | | |
| Comparative | XG/LAE 2000 | | | 4 | 4 | 2 | 2 | FAIL |
| C | ppm | | | | | | | |
| Comparative | XG/ GL-A | 0.24 | | 4 | 4 | 4 | 4 | FAIL |
| D | | | | | | | | |
| Comparative | XG/GL-A | 0.36 | | 4 | 4 | 4 | 4 | FAIL |
| E | | | | | | | | |
| Comparative | XG/ GL-L | 0.13 | | 4 | 4 | 4 | 4 | FAIL |
| F | | | | | | | | |
| Comparative | XG/GL-L/LAE | 0.05 | 0.5 | 4 | 4 | 2 | 2 | FAIL |
| G | 1000 ppm | | | | | | | |
| Comparative | XG/GL-L/LAE | 0.11 | 0.5 | 4 | 4 | 0 | 1 | FAIL |
| H | 2000 ppm | | | | | | | |
| I | XG/GL-A/LAE | 0.24 | 4.8 | 0 | 0 | 2 | 2 | PASS |
| | 500 ppm | | | | | | | |
| J | XG/GL-A/LAE | 0.51 | 10 | 0 | 0 | 2 | 1 | PASS |
| | 500 ppm | | | | | | | |
| K | XG/GL-A/LAE | 0.15 | 1.4 | 0 | 0 | 2 | 2 | PASS |
| | 1000 ppm | | | | | | | |
| L | XG/GL-A/LAE | 0.30 | 1.4 | 0 | 0 | 2 | 0 | PASS |
| | 2000 ppm | | | | | | | |
| Comparative | XG/CHD.G | | | 4 | 4 | 2 | 2 | FAIL |
| M | 1000 ppm | | | | | | | |
| N | XG/GL-A/ | 0.81 | 8.1 | 0 | 0 | 1 | 0 | PASS |
| | CHD.G | | | | | | | |
| | 1000 ppm | | | | | | | |

The unpreserved anionic xanthan gum lotion was further dosed with various antimicrobial booster compounds according to the samples and weight percentages shown in Table 5. As shown, none of the lotion formulations passed the challenge test when dosed with NaBz, GC, or HAP alone or with NaBz combined with GL-A or LAE. However, when the anionic formulations were dosed with the mixtures of LAE or CHD.G and GL-A and with the booster compounds as shown in Table 5, the formulations were provided with effective antimicrobial activity and passed the challenge criteria.

TABLE 5

| | | | Acidic | | | | | |
|---|---|---|---|---|---|---|---|---|
| | | Acidic | Glycolipids | Bacteria | | Fungi | | |
| | Preservative | Glycolipids | to /LAE or | D | D | D | D | Overall |
| Sample | Treatment | Conc. [% wt] | CHD.G Ratio | 21 | 28 | 21 | 28 | Result |
| Comparative | Xanthan Gum | | | 4 | 4 | 4 | 4 | FAIL |
| A | Lotion (XG) blank | | | | | | | |
| Comparative | XG/NaBz 0.2% | | | 4 | 4 | 4 | 4 | FAIL |
| O | | | | | | | | |
| Comparative | XG/GC 0.5% | | | 4 | 4 | 4 | 4 | FAIL |
| P | | | | | | | | |
| Comparative | XG/HAP 0.3% | | | 3 | 3 | 3 | 2 | FAIL |
| Q | | | | | | | | |
| Comparative | XG/GL-A/NaBz | 0.24 | | 4 | 4 | 4 | 4 | FAIL |
| R | 0.2% | | | | | | | |
| Comparative | XG/LAE 2000 | | | 4 | 4 | 0 | 2 | FAIL |
| S | ppm - NaBz 0.2% | | | | | | | |
| T | XG/GL-A/LAE | 0.12 | 4.8 | 0 | 0 | 0 | 0 | PASS |
| | 250 ppm - NaBz | | | | | | | |
| | 0.2% | | | | | | | |
| U | XG/GL-A/LAE | 0.24 | 4.8 | 0 | 0 | 1 | 0 | PASS |
| | 500 ppm - NaBz | | | | | | | |
| | 0.2% | | | | | | | |
| V | XG/GL-A/LAE | 0.25 | 10.2 | 0 | 0 | 0 | 0 | PASS |
| | 250 ppm - NaBz | | | | | | | |
| | 0.2% | | | | | | | |
| W | XG/GL-A/LAE | 0.69 | 9.1 | 0 | 0 | 0 | 0 | PASS |
| | 750 ppm - NaBz | | | | | | | |
| | 0.15% | | | | | | | |
| X | XG/GL-A/LAE | 0.26 | 10.2 | 0 | 0 | 2 | 2 | PASS |
| | 250 ppm - GC | | | | | | | |
| | 0.5% | | | | | | | |
| Y | XG/GL-A/LAE | 0.51 | 10.2 | 0 | 0 | 2 | 2 | PASS |
| | 500 ppm - GC | | | | | | | |
| | 0.2% | | | | | | | |
| Z | XG/GL-A/LAE | 0.51 | 10.2 | 0 | 0 | 1 | 1 | PASS |
| | 500 ppm - GC | | | | | | | |
| | 0.5% | | | | | | | |
| AA | XG/GL-A/LAE | 0.51 | 10.2 | 0 | 0 | 2 | 1 | PASS |
| | 500 ppm - GC | | | | | | | |
| | 1.0% | | | | | | | |
| BB | XG/GL-A/CHD.G | 0.81 | 8.1 | 0 | 0 | 0 | 0 | PASS |
| | 1000 ppm - 0.2% | | | | | | | |
| | NaBz | | | | | | | |
| CC | XG/GL-A/CHD.G | 0.81 | 8.1 | 0 | 0 | 0 | 0 | PASS |
| | 1000 ppm - 0.3% | | | | | | | |
| | HAP | | | | | | | |
| DD | XG/GL-A/LAE | 0.51 | 10.2 | 0 | 0 | 0 | 0 | PASS |
| | 500ppm - 0.2% | | | | | | | |
| | NaBz | | | | | | | |
| EE | XG/GL-A/LAE | 0.51 | 10.2 | 0 | 0 | 2 | 1 | PASS |
| | 500ppm - 0.3% | | | | | | | |
| | HAP | | | | | | | |

1-52. (canceled)

53. A product formulation comprising
at least one of an anionic rheology modifier, an anionic surfactant, or an anionic emulsifier; and
an antimicrobial effective amount of a combination of at least one cationic antimicrobial and at least one acidic glycolipid,
wherein the cationic antimicrobial comprises
(i) a compound having one or more cationic guanidine or amidine functional groups; or
(ii) a N^{α}-alkanoyl dibasic amino acid ester salt,
the at least one acidic glycolipid comprises at least one acidic sophorolipid, and
the amount by weight of the at least one acidic glycolipid is greater than or about equal to the amount by weight of the at least one cationic antimicrobial.

54. The product formulation according to claim 53, wherein the cationic antimicrobial comprises dodecylguanidine, chlorhexidine, alexidine, polyhexamethylene biguanide, polyaminopropyl biguanide, hexamidine, a salt thereof or a combination thereof.

55. The product formulation according to claim 53, wherein the cationic antimicrobial comprises a N^{α}-alkanoyl dibasic amino acid ester salt of the formula (I) 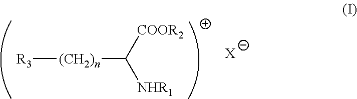
where R₁ is a straight alkyl chain from a saturated fatty acid or a hydroxy acid having from 6 to 20 carbon atoms bonded to the α-amino group through an amidic bond,
R₂ is a straight or branched alkyl chain from 1 to 6 carbon atoms, or an aromatic group,
R₃ is selected from 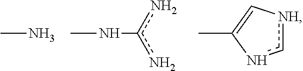
n is from 1 to 6, and
X⁻ is Cl⁻, Br⁻ or a counter ion derived from an organic or inorganic acid or a phenolic compound.

56. The product formulation according to claim 53, wherein the cationic antimicrobial comprises ethyl lauroyl arginate HCl (LAE), pyrrolidone carboxylic acid (PCA) ethyl cocoyl arginate, chlorhexidine digluconate or a combination thereof.

57. The product formulation according to claim 53, wherein the at least one acidic sophorolipid is of the formula (IV) 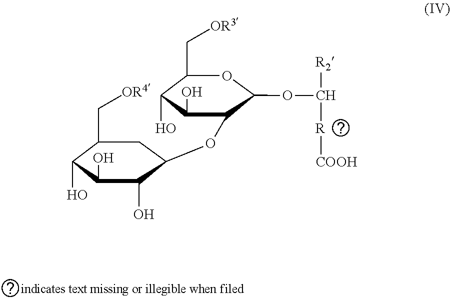
where R^{1′} is an optionally substituted saturated or unsaturated hydrocarbon chain having 8 to 20 carbon atoms, which can be linear or branched,
R^{2′} is a hydrogen atom or a saturated or unsaturated alkyl functional group having from 1 to 9 carbon atoms, which can be linear or branched, and is optionally substituted with one or more hydroxy groups; and
R^{3′} and R^{4′} are independently a hydrogen atom or an acetyl group.

58. The product formulation according to claim 53, wherein the ratio by weight of the acidic glycolipid to the cationic antimicrobial is from 1.2:1 to 20:1.

59. The product formulation according to claim 53, wherein the at least one cationic antimicrobial is present in an amount no greater than 0.5 wt %, based on the weight of the formulation.

60. A method of controlling microbial growth in or preserving a product formulation comprising at least one of an anionic rheology modifier, an anionic surfactant or an anionic emulsifier, the method comprising
adding to the product formulation an antimicrobial effective amount of at least one cationic antimicrobial and at least one acidic glycolipid,
wherein the cationic antimicrobial comprises
(i) a compound having one or more cationic guanidine or amidine functional groups; or
(ii) a N^{α}-alkanoyl dibasic amino acid ester salt,
the at least one acidic glycolipid comprises at least one acidic sophorolipid, and the amount by weight of the at least one acidic glycolipid is greater than or about equal to the amount by weight of the at least one cationic antimicrobial.

61. The method of claim 60, wherein the cationic antimicrobial comprises dodecylguanidine, chlorhexidine, alexidine, polyhexamethylene biguanide, polyaminopropyl biguanide, hexamidine, a salt thereof or a combination thereof.

62. The method of claim 60, wherein the cationic antimicrobial comprises a N^{α}-alkanoyl dibasic amino acid ester salt of the formula (I) 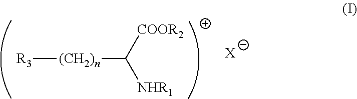
where R₁ is a straight alkyl chain from a saturated fatty acid or a hydroxy acid having from 6 to 20 carbon atoms bonded to the α-amino group through an amidic bond,
R₂ is a straight or branched alkyl chain from 1 to 6 carbon atoms, or an aromatic group,
R₃ is selected from 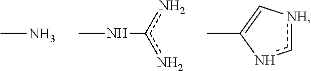
n is from 1 to 6, and
X⁻ is Cl⁻, Br⁻ or a counter ion derived from an organic or inorganic acid or a phenolic compound.

63. The method of claim 60, wherein the cationic antimicrobial comprises ethyl lauroyl arginate HCl (LAE), pyrrolidone carboxylic acid (PCA) ethyl cocoyl arginate, chlorhexidine digluconate or a combination thereof.

64. The method of claim 60, wherein the at least one acidic sophorolipid is of the formula (IV) 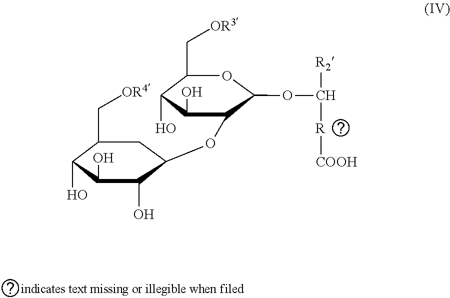
where R^{1′} is an optionally substituted saturated or unsaturated hydrocarbon chain having 8 to 20 carbon atoms, which can be linear or branched;
R^{2′} is a hydrogen atom or a saturated or unsaturated alkyl functional group having from 1 to 9 carbon atoms, which can be linear or branched, and is optionally substituted with one or more hydroxy groups; and
R^{3′} and R^{4′} are independently a hydrogen atom or an acetyl group.

65. The method according to claim 64, wherein the at least one acidic sophorolipid is present in a mixture with at least one lactonic sophorolipid, wherein at least 50% by weight, based on the total weight of sophorolipids in the mixture, are in acidic form.

66. The method of claim 60, wherein the ratio by weight of the acidic glycolipid to the cationic antimicrobial is from 1.2:1 to 20:1.

67. The method of claim 60, wherein the at least one cationic antimicrobial is added in an amount no greater than 0.5 wt %, based on the weight of the formulation.

68. An antimicrobial composition comprising from about 10 wt % to about 95 wt %, based on the total weight of the antimicrobial composition, of a combination of (a) at least one cationic antimicrobial and (b) at least one acidic glycolipid, wherein the cationic antimicrobial comprises
(i) a compound having one or more cationic guanidine or amidine functional groups; or
(ii) a N^{α}-alkanoyl dibasic amino acid ester salt, and
the at least one acidic glycolipid comprises at least one acidic sophorolipid, and the ratio by weight of (b) to (a) is from about 1:1 to 100:1.

69. The antimicrobial composition according to claim 68, wherein the cationic antimicrobial comprises a N^{α}-alkanoyl dibasic amino acid ester salt of the formula (I) 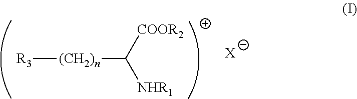
where R₁ is a straight alkyl chain from a saturated fatty acid or a hydroxy acid having from 6 to 20 carbon atoms bonded to the α-amino group through an amidic bond,
R₂ is a straight or branched alkyl chain from 1 to 6 carbon atoms, or an aromatic group,
R₃ is selected from 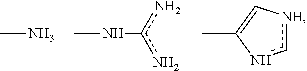
n is from 1 to 6, and
X⁻ is Cl⁻, Br⁻ or a counter ion derived from an organic or inorganic acid or a phenolic compound.

70. The antimicrobial composition according to claim 68, wherein the cationic antimicrobial comprises ethyl lauroyl arginate HCl (LAE), pyrrolidone carboxylic acid (PCA) ethyl cocoyl arginate, chlorhexidine digluconate or a combination thereof.

71. The antimicrobial composition according to claim 68, wherein the at least one acidic sophorolipid is of the formula (IV) 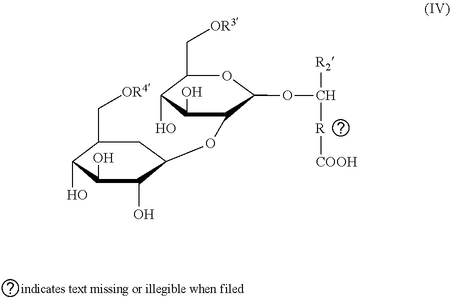
where R^{1′} is an optionally substituted saturated or unsaturated hydrocarbon chain having 8 to 20 carbon atoms, which can be linear or branched;
R^{2′} is a hydrogen atom or a saturated or unsaturated alkyl functional group having from 1 to 9 carbon, which can be linear or branched, and is optionally substituted with one or more hydroxy groups; and
R^{3′} and R^{4′} are independently a hydrogen atom or an acetyl group.

72. The antimicrobial composition according to claim 68, wherein the ratio by weight of the acidic glycolipid to the cationic antimicrobial is from 1.2:1 to 20:1.
